(19) United States
(12) Patent Application Publication
(10) Pub. No.: US 2013/0152225 A1
(43) Pub. Date: Jun. 13, 2013

(54) Plant Stress Tolerance Related Protein TaDREB4B and Encoding Gene and Use Thereof

(71) Applicants: Youzhi Ma, Beijing (CN); Zhaoshi Xu, Beijing (CN); Liancheng Li, Beijing (CN); Ming Chen, Beijing (CN)

(72) Inventors: Youzhi Ma, Beijing (CN); Zhaoshi Xu, Beijing (CN); Liancheng Li, Beijing (CN); Ming Chen, Beijing (CN)

(73) Assignee: Institute of Corp Science, Chinese Academy of Agriculture Science

(21) Appl. No.: 13/643,271

(22) PCT Filed: Sep. 10, 2010
(86) PCT No.: PCT/CN2010/001394

§ 371 Date: Feb. 7, 2013

(30) Foreign Application Priority Data
Apr. 27, 2010 (CN) 201010161640.2

(51) Int. Cl. C12N 15/82

(52) U.S. Cl. CPC C12N 15/8271
USPC 800/278; 536/23.6; 435/320.1; 435/419; 800/298; 800/320.3; 800/301; 435/252.2; 530/370

(57) ABSTRACT

Provided are a plant stress tolerance related protein TaDREB4B and encoding gene and use thereof. The TaDREB4B protein has the amino acid sequence as shown in SEQ ID NO. 1, which can be expressed under induction by drought, high salt, high temperature, low temperature, pathogenic bacteria, ABA, ethylene, JA and SA, and can specially regulate the transcriptional expression of gene comprising the DRE/CRT cis element (core sequence: CCGAC), thereby enhancing the drought resistance, salt tolerance, high temperature tolerance and resistance to pathogenic bacteria of powdery mildew of plant.

### FIELD OF THE INVENTION

The present invention relates to a plant stress tolerance related protein TaDREB4B, encoding gene thereof and use of the same.

### DESCRIPTION OF BACKGROUND

Drought, high salinity and low temperature stresses etc. are limiting factors affecting wheat growth and development. Therefore, it is one of the important tasks for genetic study and variety improvement of wheat to understand its responses to adverse conditions and signal transduction mechanism, as well as to improve stress resistance of wheat varieties. Stresses will bring a series of responses in plant, along with many physiological, biochemical and developmental changes. Understanding of reaction mechanism of plant to stress will provide scientific arguments for stress resistance genetic engineering study and applications thereof. Currently, study on plant stress resistance has gradually gone deep into cell and molecular level, which is combined with genetics and genetic engineering research to explore improvement of plant growth characteristics with biotechnology for the purpose of improving adaptive ability of plants to adversity.

Under adverse conditions such as drought, high salinity, low temperature stresses and the like, plants can make corresponding adjustments at molecular, cellular, and overall levels to reduce damages caused by the environment to the maximum extent so as to survive. Many genes are induced to express by stress, and the products thereof can not only directly participate in plant stress responses but also regulate expressions of other related genes or participate in signaling pathway, allowing plants to avoid or reduce damages so as to enhance resistance to stress environment. Stress-related gene products can be divided into two categories: the first category of gene-encoded products include gene products directly participating in plant stress responses such as ion channel protein, aquaporin, osmotic regulation factor (sucrose, proline and betaine etc.) synthase and the like; the second category of gene-encoded products include protein factors involving in stress-related signal transfer and gene regulation and expression, such as protein kinases, transcription factors, and the like. Among them, transcription factors play an important role in gene expression and regulation in plant stress responses.

Transcription factor, also known as trans-acting factor, is a DNA-binding protein capable of specifically acting with the cis-acting element in the promoter region of a eukaryotic gene. Interactions between transcription factors and between transcription factor and other related proteins activate or inhibit transcription. The DNA binding region of a transcription factor determines its binding specificity with the cis-acting element, and the transcriptional regulation region determines whether it functions to activate or inhibit the gene expression. In addition, its activity is further influenced by nuclear localization, oligomerization and similar effects.

The presently known stress-related transcription factors in plant mainly include: AP2 (APETALA2)/EREBP (ethylene responsive element binding protein) transcription factor family having a AP2 domain, bZIP (basic region/leucine zipper motif transcription factors)-like transcription factors having a basic region and a leucine zipper, WRKY transcription factor family containing a conservative WRKY amino acid sequence, MYC family containing a basic helix-loop-helix (bHLH) and a leucine zipper and MYB family having a tryptophan cluster (Trp cluster). Except that the WRKY family does not participate in water stress responses of plant, other four families of these five transcription factor families each involves in regulation of plant stress responses to drought, high salinity, low temperature and the like. Wherein, the AP2/EREBP-like transcription factors, widespread in higher plants, are a class of transcription factors unique to plant, which, in recent years, have been reported in Arabidopsis, tobacco, maize, rice, soybean and canola, suggesting that the AP2/EREBP-like transcription factors are widespread in higher plants and have an important role.

DREB (dehydration response element binding protein, DRE-binding protein) transcription factors are members of the EREBP-like subfamily of the AP2 family. DREB and EREBP-like transcription factors have no dramatic identity in amino acid sequences; however, both of them comprise a highly conservative DNA binding region (EREBP/AP2 domain) consisting of about 58 amino acids. Three-dimensional analysis of protein showed that this region contains three β-sheets, which play a critical role in identifying various cis-acting elements. Wherein, difference between the two amino acid residues at positions 14 and 19 in the second β-sheet determines the specific binding of these transcription factors to different cis-acting elements. In DREB-like transcription factors, the amino acid at position 14 is a valine (V14) and the amino acid at position 19 is a glutamic acid (E19), wherein the amino acid at position 19 is not conservative; for example, the amino acid at position 19 of the OsDREB1 transcription factor of rice is a valine (Dubouzet J G, Sakuma Y, Ito Y, Kasuga M, Dubouzet E G, Miura S, Seki M, Shinozaki K, Yamaguchi-Shinozaki K, 2003). V14 plays an more obviously important role than E19 in terms of determining DNA binding specificity in DREB related proteins (Sakuma Y, Liu Q, Dubouzet J G, Abe H, Shinozaki K and Yamaguchi-Shinozaki K, 2002); while in ERF transcription factors, the amino acid at position 14 is a glycine and the amino acid at position 19 is an aspartic acid, thereby, DREB specifically binds DRE/CRT cis element and ERF specifically binds GCC-box. The C-terminal region of the AP2/EREBP domain further contains a core sequence consisting of 18 amino acid residues. This core sequence forms an amphiphilic α-helix, which may be involved in interactions with other transcription factors and DNA.

At present, transcription factors containing EREBP/AP2 domain are found in many plants, which are respectively associated with signal transfer of disease resistance, stress tolerance and the like (Liu Qiang, Zhao Nan-ming, Yamaguchi-Shinozaki K, Shinozaki K, 2000). Liu Qiang et al. thought that one DREB gene may regulate expressions of multiple functional genes associated with drought, high salinity and low temperature tolerances of plan (Liu Qiang, Zhao Nanming, Yamaguchi-Shinozaki K, Shinozaki K, 2000). Investigations conducted by Kasuga et al. demonstrate that the DREB1A gene introduced into Arabidopsis may simultaneously promote expressions of stress tolerance related genes, rd29, rd17, kin1, cor6.6, cor15a and erd10, greatly enhancing the stress resistance of transgenic plants (Kasuga M, Liu Q, Miura S, Yamaguchi-Shinozaki K, Shinozaki K., 1999). Similarly, a transgenic plant of low temperature tolerance transcription factor CBF1 is significantly improved in low temperature tolerance ability (Jaglo-Ottosen K R, Gilmour S J, Zarka D G, Schabenberger O, Thomashow M F., 1998). Since stress tolerance of a plant is a complex trait regulated by multiple genes, it is very difficult to achieve comprehensive improvement of stress resistance of the plant depending on the introduction of a single functional protein gene. Therefore, it has become a research hotspot of plant stress resistance gene engineering to employ a key transcription factor to promote expressions of multiple functional genes to thereby enhancing the stress resistance of plants.

Based on the number of DNA binding regions contained, AP2/EREBP transcription factors can be classified into three major types, the AP2 (APETALA2) and ethylene responsive element binding protein EREBP (ethylene-responsive element binding protein) as well as RAV. The AP2-type transcription factors include AP2 and ANT of Arabidopsis as well as Glossy, and idsl of maize and the like. This type of transcription factor contains two AP2/EREBP domains, regulating cell growth and development. 14 AP2-type transcription factor genes have been found in Arabidopsis. The EREBP-type transcription factor contains only one AP2/EREBP domain, regulating molecular responses of plants to hormones (ethylene), pathogens, low temperature, drought and high salinity etc. In the EREBP-type transcription factors, many members such as tobacco EREBP1-4, tomato Pti4-6, Arabidopsis RAV1-2, AtEBP, AtERF1-5, DREB1A-C (CBF1-3) and DREB2A-B etc. have been found, which are respectively associated with signal transfer of cell development, hormone, disease resistance, low temperature as well as drought, high salinity and the like. These EREBP-type transcription factors can be further divided into: EREBP (ethylene-responsive element binding protein, i.e., ERF) subgroup, including tobacco EREBP1-4, tomato Pti4-6, Arabidopsis AtEBP, AtERF1-5, which specifically bind with the GCC-box containing a core sequence of AGCCGCC; therefore, the DNA binding region thereof is also referred to as the GCC-box binding domain (GBD) collectively, wherein the second G, the fifth G and the seventh C play an important role in identifying the ERF protein (Hao D, Ohme-Takagi M, Sarai A, 1998). Studies on its three dimensional structure with nuclear magnetic resonance showed that GBD of AtERF1 binds with the major groove of its target sequence, the GCC-box, by forming three reverse β-sheets; DREBP subgroup, including Arabidopsis DREB1A-C (CBF1-3) and DREB2A-B, which specifically bind with the drought response element, DRE/CRT, under drought, high salinity and low temperature. There are 124 DREBP-type transcription factor genes found in the genome of Arabidopsis; the RAV-type transcription factors include Arabidopsis RAV1, RAV2, containing two different DNA binding regions, ERF/AP2 and B3. Six RAV-type transcription factor genes have been found in Arabidopsis. There is also a special class of transcription factor, AL079349, which is a different category distinct from the above transcription factors.

Recently, it is found that EREBP proteins are involved in drought, high salinity and low temperature stress signaling as well as gene expression and regulation. Mine et al. isolated from potato tubes stored at low temperature the EREBP transcription factor CIP353, which is strongly expressed under induction by low temperature stress (Mine T, Hiyoshi T, Kasaoka K, Ohyama A, 2003), indicating that EREBP protein may possibly involve in gene expression and regulation stressed by low temperature. Park et al. obtained the EREBP transcription factor Tsi gene expressed through induction of high salinity, ethylene or jasmonic acid, with tomato as the material, and analysis by EMSA (Electrophoretic mobility shift assays) test found that Tsi protein is capable of binding with both GCC-box and DRE/CRT cis element (Park J M, Park C J, Lee S B, Ham B K, Shin R, Paek K H, 2001), though binding capacity of the former is higher than that of the latter, demonstrating that some EREBP proteins are able to activating genes expressed by induction of osmotic stress. Under normal growth conditions, over-expression of Tsi gene enhanced salinity tolerance and disease resistance of transgenic plant (35S::Tsi1) (Park J M, Park C J, Lee S B, Ham B K, Shin R, Paek K H, 2001), indicating that Tsi gene may participate in two signaling pathways of biological stress and abiotic stress. A MAPK-like signal transfer mode (including SIMKK and SIMK) activated by high salinity stress transfers the stress signal to EIN2 (downstream of CTRL of ethylene signal transfer pathway) (Guo H W and Ecker J, 2004), and finally activates some EREBP transcription factors to regulate expressions of osmotic stress-related genes and improve salinity tolerance of plants. With regard to whether there is a gene containing a GCC-box element, whose expression product directly involves in abiotic stress responses, it needs to be further confirmed.

Based on the results of current studies, plant has at least the following six signal transfer pathways under stress conditions: (1) three ABA-dependent signal transfer pathways: induced by drought and high salinity to activate MYB and MYC-like transcription factor genes, regulating target genes having a MYBR or MYCR cis-acting element; induced by drought and high salinity to activate ABF/AREB-like transcription factor genes, regulating target genes having a ABRE cis-acting element; induced by drought and high salinity to activate CBF4 and DREB1-like transcription factor genes, regulating target genes having a DRE/CRT cis-acting element. (2) three ABA-independent signal transfer pathways: induced by drought and high salinity to activate DREB2-like transcription factor genes, regulating target genes having a DRE/CRT cis-acting element; induced by low temperature to activate CBF1-3/DREB1A-C-like transcription factor genes, regulating target genes having a DRE/CRT cis-acting element; induced by drought and high salinity or induced by ethylene to activate ERF-like transcription factor genes, regulating target genes having a DRE/CRT or GCC cis-acting element.

### DISCLOSURE OF THE INVENTION

The object of the present invention is to provide a plant stress tolerance related protein, TaDREB4B, as well as encoding gene and applications thereof.

The protein provided by the present invention is a dehydration response element binding protein originating from Triticum aestivum L., which is the following (a) or (b):

(a) a protein, consisting of the amino acid sequence as shown in SEQ ID NO. 1 in the Sequence Listing;

(b) a protein, which is derived from SEQ ID NO. 1 by subjecting the amino acid sequence of SEQ ID NO. 1 to substitution and/or deletion and/or addition of one or more amino acid residues, and which is related to plant stress tolerance.

The protein as shown in SEQ ID NO. 1 consists of 346 amino acid residues, wherein amino acid residue sequences at positions 26-33 and positions 63-67 starting from the amino terminal are two possible nuclear localization signal regions, and amino acid residue sequence at positions 89-147 starting from the amino terminal is a conservative AP2/EREBP domain.

A tag as set forth in Table 1 may be linked to an amino terminal or carboxyl terminal of the protein consisting of amino acid sequence set forth by SEQ ID NO. 1 in the Sequence Listing for convenient purification of the TaDREB4B in (a).

TABLE 1
Sequence of Tags

| Tags | Residues | Sequence |
|---|---|---|
| Poly-Arg | 5-6 (typically, 5) | RRRRR |
| Poly-His | 2-10 (typically, 6) | HHHHHH |
| FLAG | 8 | DYKDDDDK |
| Strep-tag II | 8 | WSHPQFEK |
| c-myc | 10 | EQKLISEEDL |

The above described TaDREB4B in (b) may be obtained by artificial synthesis, or may be obtained by firstly synthesizing the encoding gene thereof, and then biological expression. The encoding gene of the above described TaDREB4B in (b) may be obtained by subjecting the DNA sequence as set forth by SEQ ID NO. 2 in the Sequence Listing to deletion of codons of one or more amino acid residues, and/or subjecting one or more base pairs to missense mutation, and/or linking the encoding sequence of a tag as set forth in Table 1 at the 5′ terminal and/or 3′ terminal thereof.

The gene encoding the protein also falls into the protection scope of the present invention.

The gene may be a DNA molecule of any of the following 1) or 2) or 3) or 4) or 5):

1) a DNA molecule as set forth by the nucleotides at positions 128-1168 starting from the 5′ end of SEQ ID NO. 2 in the Sequence Listing;

2) a DNA molecule as set forth by the nucleotides at positions 128-1193 starting from the 5′ end of SEQ ID NO. 2 in the Sequence Listing;

3) a DNA molecule as set forth by SEQ ID NO. 2 in the Sequence Listing;

4) a DNA molecule, which hybridizes with the DNA sequence defined in 1) or 2) or 3) under stringency conditions, and which encodes a stress tolerance related protein;

5) a DNA molecule, encoding a stress tolerance related protein, with more than 90% homology to the DNA sequence defined in 1) or 2) or 3).

The above stringency conditions is: hybridizing at 65 in a solution of 0.1×SSPE (or 0.1×SSC), 0.1% SDS and washing the membrane.

The cDNA sequence as shown in SEQ ID NO. 2 consists of 1494 nucleotides, which has an open reading frame of nucleotides at positions 128-1168 starting from the 5′ end.

The recombinant expression vector, expression cassette, transgenic cell line or recombinant strain containing the gene each falls into the protection scope of the present invention.

The recombinant expression vector containing said gene may be constructed with existing plant expression vectors. The plant expression vector includes binary Agrobacterium vectors, vectors that can be used in plant microprojectile bombardment and the like. The plant expression vector may further includes a non-translational region of the 3′ terminal of a exogenous gene, that is, includes a polyadenylic acid signal and any other DNA fragments involved in mRNA processing or gene expression. The polyadenylic acid signal may guide addition of a polyadenylic acid to the 3′ terminal of a pre-mRNA, for example, the non-translational regions transcribed from the 3′ terminals of plasmid genes (e.g., nopaline synthetase Nos gene), and plant genes (e.g., soybean storage protein gene) under the induction of the crown-gall nodule of Agrobacterium have similar functions. When constructing a recombinant plant expression vector using said gene, any one of enhancement promoters or constitutive promoters which can be used alone or in combination with other plant promoters, such as 35S promoters of cauliflower mosaic virus (CAMV), ubiquitin promoters of maize, may be added before the transcription initiation nucleotide of the gene; in addition, when constructing a plant expression vector using the gene of the present invention, an enhancer including translational enhancers or transcription enhancers may also be used. These enhancer regions may be ATG start codon or start codon of an adjacent region and the like, which must be identical with the reading frame of a coding sequence to guarantee the correct translation of the whole sequence. There are abundant sources for the translation regulatory signal and start codon, which may be natural-occurring or synthesized. A translation initiation region may be from a transcription initiation region or a structural gene. The plant expression vector to be used may be processed, for example, by introducing a gene encoding a color-changeable enzyme or a luminous compound (GUS gene, luciferase gene etc.), an antibiotic marker with resistance (gentamicin marker, kanamycin marker etc.) or a marker gene for an anti-chemical reagent (such as herbicide-resistant gene) and the like that may be expressed in a plant, for convenient identification and screening of a transgenic plant cell or a plant. In consideration of the safety of transgenic plant, a transformed plant may be directly screened under stress without introducing any selective marker gene.

More particularly, the recombinant expression vector may be YEP-GAP-TaDREB4B, pBI121-TaDREB4B or pAHC25-TaDREB4B.

The YEP-GAP-TaDREB4B is a recombinant plasmid obtained by inserting said gene into a multiple cloning site of YEP-GAP. The YEP-GAP-TaDREB4B is preferably a recombinant plasmid obtained by inserting a DNA fragment as set forth by nucleotides at positions 128-1193 starting from the 5′ end of SEQ ID NO. 2 in the Sequence Listing between recognition sites of EcoRI and XhoI of YEP-GAP.

The pBI121-TaDREB4B is a recombinant plasmid obtained by inserting said gene into a multiple cloning site of pBI121. The pBI121-TaDREB4B is preferably a recombinant plasmid obtained by inserting a DNA fragment as set forth by nucleotides at positions 128-1193 starting from the 5′ end of SEQ ID NO. 2 in the Sequence Listing between recognition sites of BamHI and XhoI of pBI121.

The pAHC25-TaDREB4B is a recombinant plasmid obtained by inserting said gene into a multiple cloning site of pAHC25. The pAHC25-TaDREB4B is preferably a recombinant plasmid obtained by inserting a DNA fragment as set forth by nucleotides at positions 128-1193 starting from the 5′ end of SEQ ID NO. 2 in the Sequence Listing between recognition sites of SmaI and SpeI of pAHC25.

The present invention further claims a method for cultivating a transgenic plant, which introduces the gene into a target plant to give a transgenic plant with stress tolerance higher than that of the target plant. More particularly, the gene may be introduced into the target plant via the recombinant expression vector. An expression vector bearing said gene may transform a plant cell or tissue by using conventional biological methods such as Ti plasmid, Ri plasmid, plant virus vector, direct DNA transformation, microinjection, conductivity method, Agrobacterium tumefaciens mediation and so on, and the transformed plant tissue may be cultivated into a plant.

The stress tolerance may be abiotic stress tolerance or disease resistance.

More particularly, the abiotic stress tolerance may be drought tolerance and/or salinity tolerance and/or high temperature tolerance (such as 43).

The target plant may not only be a monocotyledonous plant but also a dicotyledonous plant, such as Arabidopsis (such as Arabidopsis (Columbia ecotype)), wheat (such as Jimai 19) and the like.

Said drought tolerance may be reflected as the following (I) and/or (II):

(I) the transgenic plant has a proline content and/or a content of total soluble sugar and/or a peroxidase activity and/or a photosynthetic rate higher than that of the target plant;

(II) the transgenic plant has a grain weight of plant and/or a 1000-grains weight higher than that of the target plant under drought conditions.

Said disease resistance may be a resistance to powdery mildew, more particularly, may be a resistance to the powdery mildew caused by the powdery mildew pathogenic bacteria E09.

The present invention further claims use of the protein as a transcription factor.

### BRIEF DESCRIPTION OF THE DRAWINGS

FIG. 1 shows the results of homology alignment between TaDREB4B and TaDREB amino acid sequence of wheat.

FIG. 2 shows a real time PCR map of TaDREB4B expressed under the induction of stress; A: treated with abscisic acid; B: treated with ethylene; C: treated with high temperature; D: treated with methyl jasmonate; E: treated with chilling injury; F: treated with salt; G: treated with drought; H: treated with salicylic acid; I: treated with pathogenic bacteria of powdery mildew.

FIG. 3 is a schematic map showing that the yeast one-hybrid system proves the principle of the binding specificity in vivo and activation properties of a transcription factor.

FIG. 4 is a comparison of drought resistance between the wild type and transgenic Arabidopsis.

FIG. 5 is a comparison of salinity tolerance between the wild type and transgenic Arabidopsis.

FIG. 6 is a comparison of high temperature tolerance between the wild type and transgenic Arabidopsis.

FIG. 7 is a comparison of drought resistance indexes between the wild type and transgenic wheat; A: proline content; B: content of total soluble sugar; C: POD enzyme activity; D: SPAD value.

FIG. 8 is a comparison of resistance to the pathogenic bacteria of powdery mildew between the wild type and transgenic wheat.

### BEST MODE FOR CARRYING OUT THE INVENTION

The following examples are presented for purposes of better understanding the present invention, which, however, are not intended to limit the present invention. Each of the experimental methods in the following examples is a conventional method, unless otherwise indicated. All of the experimental materials used in the following examples are obtained from conventional Biochemical Reagent Shops, unless otherwise indicated. Each of the % in the following examples denotes weight percentage content, unless otherwise indicated.

### Example 1

### Cloning of TaDREB4B

I. Isolation of mRNA

Seedlings of Xiaobaimai (National Germplasm Repository of China, Number ZM242), at 3-leaf stage, cultured in water for about 10 days were treated with drought for 2 h, quickly frozen with liquid nitrogen, and stored at −80° C. fur use. Then, mRNA was isolated with Quikprep Micro mRNA Purification Kit (Pharmacia).

II. Construction of cDNA Library and Titer Assay
1. Construction of cDNA Library

cDNA double strand were synthesized with the mRNA obtained from step I by using Timesaver™ cDNA Synthesis Kit (Pharmacia), to which a EcoRI/NotI adaptor was added; construction of cDNA library was conducted by using ZAP Express® Predigested Gigapack® III Gold Cloning Kit (Stratagene), giving a total of 500 ul library liquid.

### 2. Assay of Titer

(1) 1 ul of library liquid was taken and diluted 1000-fold with SM Buffer;

(2) 1 ul, 10 ul and 100 ul of the diluents were separately taken to three 10 ml centrifuge tubes, to which, 100 ul of competent host bacteria, XL1-Blue MRF′ (OD₆₀₀, 1.0), was added respectively, and incubated at 37° C. for 20 min;

(3) to each tube, 3 ml top gel (50° C.) was added and mixed, immediately after that, the mixture was spread onto a solid NZY plate, which was placed upside down after solidification, and cultivated at 37° C. overnight;

(4) based on plaque number on the plate, average value was calculated, that is, the library capacity.

$$Calculation formula \text{:}$$

$$\frac{Number of plaque ( pfu ) \times dilution factor}{Volume of phage diluent ( ul )} \times 1000 ul \text{/} ml$$

After calculation, this cDNA library has a titer of 3.0×10⁶ plaques.

III. Screening of cDNA Library

### 1. Preparation of Probe

Primers, WAPF and WAPR, were designed based on the sequence of AP2 conserved region of cloned DREB gene, and PCR amplification was conducted with cDNA of common wheat as the template.

| | |
|---|---|
| WAPF: | 5′-ACC GCG GTG TGA GGC AGA GGA-3′; |
| WAPR: | 5′-TGA GAA GTT GAC ACG TGC TTT GGC-3′. |

PCR amplification products were identified via a 1.2% agarose gel electrophoresis.

### 2. Recovery of Probe

Bands of 180 bp (probes) were recovered with Agarose Gel DNA Purification Kit Ver.2.0 (TaKaRa Company, Code No.: DV805A).

### 3. Film Transfer

(1) 1 ul library liquid of 1 of step II was taken into a Petri dish to cultivate phages to about 6.0×10³ pfu;

(2) plaques were cooled at 4° C. after cultivation, which was taken out immediately before use, placed on a superclean bench and blown to dry to prevent sticking of the top gel by the film during film transferring;

(3) a Hybrond-N⁺ film was cut into a round shape, slightly smaller than the Petri dish having a diameter of 150 mm; code and date were marked on the film with a pencil (corresponding to the Petri dish);

(4) two edges of the film were clamped by a forceps, with the side being marked upward; the film was firstly contacted the plate with its middle portion, and then slowly released until naturally flattened, be sure not to move the film and keep bubbles away; finally, the film was completely flattened before timing;

(5) the film was pierced three asymmetric holes with a syringe needle, and the Petri dish was marked with a marker pen at corresponding positions on its backside;

(6) after 3 min, the film was gently lifted by the forceps from one edge without sticking the top gel;

(7) the film was quickly placed into a Petri dish filled with a denaturing solution (one layer of filter paper and 15 ml denaturing solution were placed in the Petri dish), denaturing for 7 min, with the side being marked downward; be sure not to let the solution reach the upper surface of the film;

(8) the film was transferred to a Petri dish filled with a neutralization solution (one layer of filter paper and 15 ml neutralization solution were placed in the Petri dish), neutralizing twice, each for 3 min;

(9) the film was then transferred into a washing solution, washing for 30 min, which may be gently shaken at the same time;

(10) the film was removed and dried on a clean filter paper, with the side being marked downward;

(11) the film was wrapped with a plastic wrap, crosslinked on a UV crosslinker for 1 min, and stored at 4° C. for use.

### 4. Pre-Hybridization and Hybridization Reaction

Pre-hybridization was performed at 65° C. for 5-6 h (new film); after labeling with probes, a NaOH solution was added; the mixture was mixed, stood at room temperature for 10 min so as to denature the probe, and then hybridized at 65° C. overnight.

### 5. Elution

The film was washed at 55° C.-65° C., sucked to dry with a filter paper, wrapped with a plastic wrap, and pressed to give an X-ray film.

### 6. Second Screening of Positive Clones

(1) The X-ray film was aligned with the film to determine its location, on which positions of the three asymmetric holes in the film was drawn;

(2) the X-ray film and corresponding Petri dish were placed on a film reader to locate the Petri dish based on the asymmetric dots;

(3) the confirmed positive hybridized plaques were taken, by a 1 ml gun tip with the head being removed, into a 1 ml SM buffer solution, to which 50 ul chloroform was added;

(4) the solution was oscillated for 30 sec, placed at room temperature for 1 h, and centrifuged so as to collect the supernatant;

(5) 10-50 ul supernatant was taken to spread the plate again, cultivating phages for second screening;

(6) the second screening comprised the same steps as described above: film transfer, pre-hybridization and hybridization reaction, elution, pressing X-ray film, and obtaining of individual positive plaques.

### IV. Obtaining of TaDREB4B

### 1. Mass Excision

(1) Preparation of XL1-Blue MRF′ and XLOLR strain; XL1-Blue MRF′ and XLOLR strain were cultivated with liquid LB medium and placed at 30° C. overnight; the medium was supplied with 0.2% maltose, 10 mM MgSO₄ and antibiotics, which were 12.5 μg/ml tetracycline and 50 μg/ml kanamycin, respectively; on the second day, a 1000×g centrifugation was conducted for 10 min to collect strains, which were re-suspended with 10 mM MgSO₄, enabling the OD₆₀₀ to reach 1.0;

(2) a 10 ml sterile centrifuge tube was added with:

| | |
|---|---|
| 1 μl library liquid | (containing about 6.0 × 10³ phage particles) |
| XL1-Blue MRF′ | 200 μl (OD₆₀₀, 1.0) |
| ExAssist helper phage | 2 μl (>1 × 10¹⁰ pfu/ml) |

(3) incubated at 37° C. for 15 min;

(4) added with 20 ml liquid NZY medium, oscillated at 37° C. to cultivate for 2.5-3h;

(5) heated at 65-70° C. for 20 min;

(6) centrifuged at 1000×g for 10 min, and the supernatant was moved into a new tube;

(7) 200 μl XLOLR strain and 1 μl supernatant were mixed in a 1.5 ml centrifuge tube;

(8) incubated at 37° C. for 15 min;

(9) 10 μl, 100 μl bacteria solutions were spread onto a LB solid medium (containing 50 μg/ml ampicillin), respectively, and cultivated at 37° C. overnight.

2. Inspection of Insert Fragments of cDNA Library

(1) Mono-colonies excised from the mass in step 1 were randomly selected, from which plasmid DNA was extracted;

(2) the plasmid DNA was digested with restriction endonuclease EcoRI (Takara) using the following reaction system, 10 μl:

| | |
|---|---|
| 10 × buffer H | 1 μl |
| EcoRI (12U/μl) | 0.5 μl |
| plasmid DNA | 2 μl |
| ddH₂O | 6.5 μl |

(3) the digestion was performed at 37° C. for 2 h; insert fragments were found present in more than 95% of the vectors via a 0.8% agarose gel electrophoresis, indicating that more than 95% of the phages contain a recombinant; therefore, the library actually comprises 2.85×10⁶ recombinants (the cDNA library has a titer of 3.0×10⁶). More than 50% of the recombinants contain an insert fragment of 800 bp-4 Kb in length, suggesting that the constructed library is complete.

### 3. Single-Clone Excision

(1) The obtained individual positive plaques were dug out from the plate, put into a sterile centrifuge tube added with 500 μl SM buffer and 20 μl chloroform, subjected to a vortex oscillation for 10 sec, and stored at 4° C.;

(2) XL1-Blue MRF′ and XLOLR strain were cultivated with a liquid LB medium, stood at 30° C. overnight; the medium was supplied with 0.2% (w/v) maltose, 10 mM MgSO₄ and antibiotics, which were 12.5 μg/ml tetracycline and 50 μg/ml kanamycin, respectively;

(3) on the second day, a 1000×g centrifugation was conducted for 10 min to collect strains, which were re-suspended with 10 mM MgSO₄, enabling the OD₆₀₀ to reach 1.0;

(4) a 10 ml sterile centrifuge tube was added with:

| | |
|---|---|
| XL1-Blue MRF′ | 200 μl (OD₆₀₀, 1.0) |
| phage stock solution | 250 μl (containing at least 1 × 10⁵ phage particles) |
| ExAssist helper phage | 1 μl (>1 × 10¹⁰ pfu/ml) |

(5) incubated at 37 for 15 min;

(6) added with 3 ml liquid NZY medium, oscillated at 37° C. to cultivate for 2.5-3h;

(7) the centrifuge tube was placed in a water bath at 65-70° C. for 20 min, and then, centrifuged at 1000×g for 15 min;

(8) the supernatant was moved into a new centrifuge tube, that is, the phagemid suspension;

(9) into a 1.5 ml centrifuge tube, the XLOLR strain prepared in step (3), 200 μl, and the phagemid suspension prepared in step (8), 100 μl, were added, followed by liquid NZY medium, 300 μl; the mixture was incubated at 37° C. for 45-60 min;

(10) 50 μl bacteria solution was spread onto a LB solid medium (containing 50 μg/ml ampicillin), and cultivated at 37° C. overnight;

(11) the positive clones were picked out on the second day, cultivated in a liquid LB medium overnight, from which, plasmids were extracted, digested with EcoRI and detected for length of the insert fragments by via an electrophoresis.

(12) Clones having an insert fragment of longer than 800 bp were selected for sequencing, which was conducted on a ABI733 sequencer (Genecore Biological Company) with a dideoxynucleotide chain termination method; the resulting full sequences were compared with sequences from nucleotide data bases such as EMBL Bank, GENEBANK and the like, and analyzed with the DNASIS software. As a result, clone 18 was found to have a conservative AP2/EREBP domain; moreover, structure of the gene was complete.

(13) The nucleotide sequence represented by SEQ ID NO. 2 in the Sequence Listing was obtained by analysis of the nucleotide sequence of clone 18 and corresponding amino acid sequence.

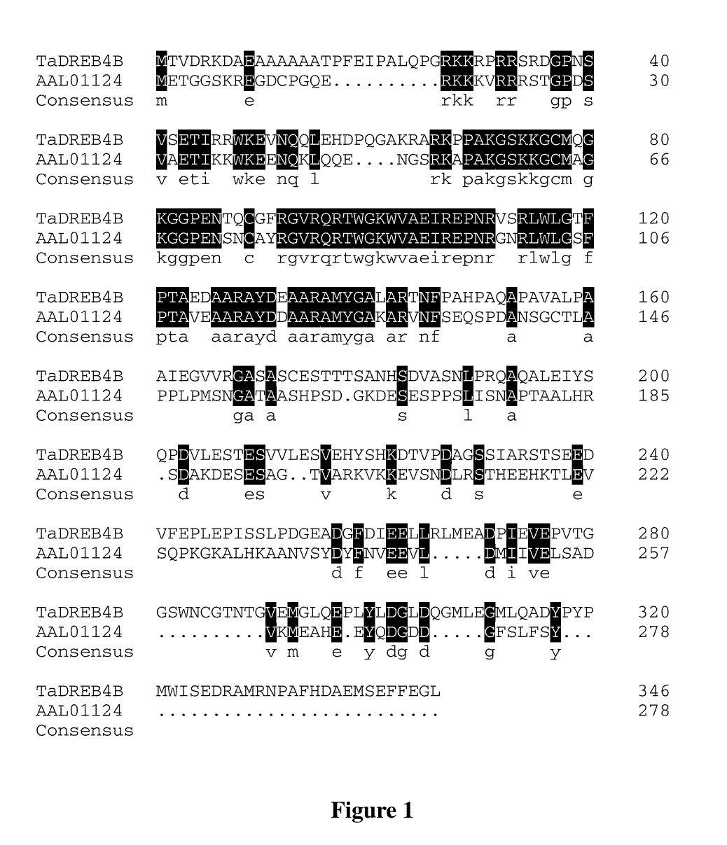

The protein represented by SEQ ID NO. 1 in the Sequence Listing, designated as TaDREB4B protein, is consisted of 346 amino acid residues. In SEQ ID NO. 1, amino acid residues at positions 26-33 and positions 63-67 starting from the amino terminal are two possible nuclear localization signal regions, and amino acid residues at positions 89-147 starting from the amino terminal is a conservative AP2/EREBP domain. As shown in FIG. 1 (black box denotes identical amino acid portions), the result of homologous sequences alignment of TaDREB4B protein demonstrates that TaDREB4B only has a homology of 34.97% to the previously reported wheat TaDREB (AAL01124), indicating that TaDREB4B is a newly discovered wheat protein. The encoding gene of TaDREB4B protein, designated as TaDREB4B gene, has an open reading frame of nucleotides at positions 128-1168 starting from the 5′ end of SEQ ID NO. 2 in the Sequence Listing.

### Example 2

### Analysis of Expression Properties of TaDREB4B with Real Time Quantitative PCR

I. Various Stress Treatments were Conducted

10-day old seedlings of Xiaobaimai were subjected to the following treatments.

(1) Drought treatment: wheat seedlings cultured in water were taken out, moistures on roots were sucked dry, placed onto a dry filter paper, and cultivated in drought conditions for 30 min, 1 h, 2 h, 4 h, 8 h, 12 h, 24 h before sampling materials; the materials were quickly frozen with liquid nitrogen and stored at −80° C. fur use.

(2) Salt treatment: wheat seedlings were put into a 2% sodium salt solution consisted of NaCl and Na₂SO₄ (the mass percentage ratio of NaCl to Na₂SO₄ was 3:2) and cultivated under light for 30 min, 1 h, 2 h, 4 h, 8 h, 12 h, 24 h before sampling materials, respectively; the materials were quickly frozen with liquid nitrogen and stored at −80° C. fur use.

(3) Abscisic acid treatment: wheat seedlings were put into an aqueous solution of 200 μM abscisic acid (ABA), and cultivated under light for 30 min, 1 h, 2 h, 4 h, 8 h, 12 h, 24 h before sampling materials, respectively; the materials were quickly frozen with liquid nitrogen and stored at −80° C. fur use.

(4) Treatment with pathogenic bacteria of powdery mildew: wheat seedlings were inoculated with powdery mildew strains, and cultivated under light for 3 h, 6 h, 12 h, 2d, 3d, 4d, 5d before sampling materials; the materials were quickly frozen with liquid nitrogen and stored at −80° C. fur use.

(5) Chilling injury treatment: wheat seedlings were placed in a 4° C. incubator and cultivated under light for 30 min, 1 h, 2 h, 4 h, 8 h, 12 h, 24 h before sampling materials, respectively; the materials were quickly frozen with liquid nitrogen and stored at −80° C. fur use.

(6) Methyl jasmonate treatment: wheat seedlings were put into a solution of 50 μM methyl jasmonate (JA), and cultivated under light for 30 min, 1 h, 2 h, 4 h, 8 h, 12 h, 24 h before sampling materials, respectively; the materials were quickly frozen with liquid nitrogen and stored at −80° C. fur use.

(7) Ethylene treatment: wheat seedlings were put into a plastic bag containing ethylene, and cultivated under light for 30 min, 1 h, 2 h, 4 h, 8 h, 12 h, 24 h before sampling materials, respectively; the materials were quickly frozen with liquid nitrogen and stored at −80° C. fur use.

(8) Salicylic acid treatment: wheat seedlings were put into a solution of 50 μM salicylic acid (SA), and cultivated under light for 30 min, 1 h, 2 h, 4 h, 8 h, 12 h, 24 h before sampling materials, respectively; the materials were quickly frozen with liquid nitrogen and stored at −80° C. fur use.

(9) High temperature treatment: wheat seedlings were placed in a condition at 42° C., and cultivated under light for 30 min, 1 h, 2 h, 4 h, 8 h, 12 h, 24 h before sampling materials, respectively; the materials were quickly frozen with liquid nitrogen and stored at −80° C. fur use.

(10) Control treatment: materials were taken directly from wheat seedlings without any treatment, and stored at −80° C. for using as a control (Oh).

II. Isolation of mRNA

mRNA was isolated by using Quikprep Micro mRNA Purification Kit (Pharmacia).

III. Reverse Transcribed to cDNA

cDNA was reverse transcribed from the purified mRNA using R103-Quant_Reverse_Transcriptase (TIANGEN).

### IV. Real Time Fluorescence Quantitative PCR

Specific primers, TaDREB4BRTF and TaDREB4BRTR, were designed based on the variable region of TaDREB4B sequence. Actin was used as an internal reference gene with primers, actin-2F and actin-2R.

| | |
|---|---|
| TaDREB4BRTF: | 5′-GATGTGTTCGAGCCATTGGAG-3′; |
| TaDREB4BRTR: | 5′-TGGTCCAAGCCATCCAGGTAG-3′. |
| actin-2F: | 5′-CTCCCTCACAACAACCGC-3′; |
| actin-2R: | 5′-TACCAGGAACTTCCATACCAAC-3′. |

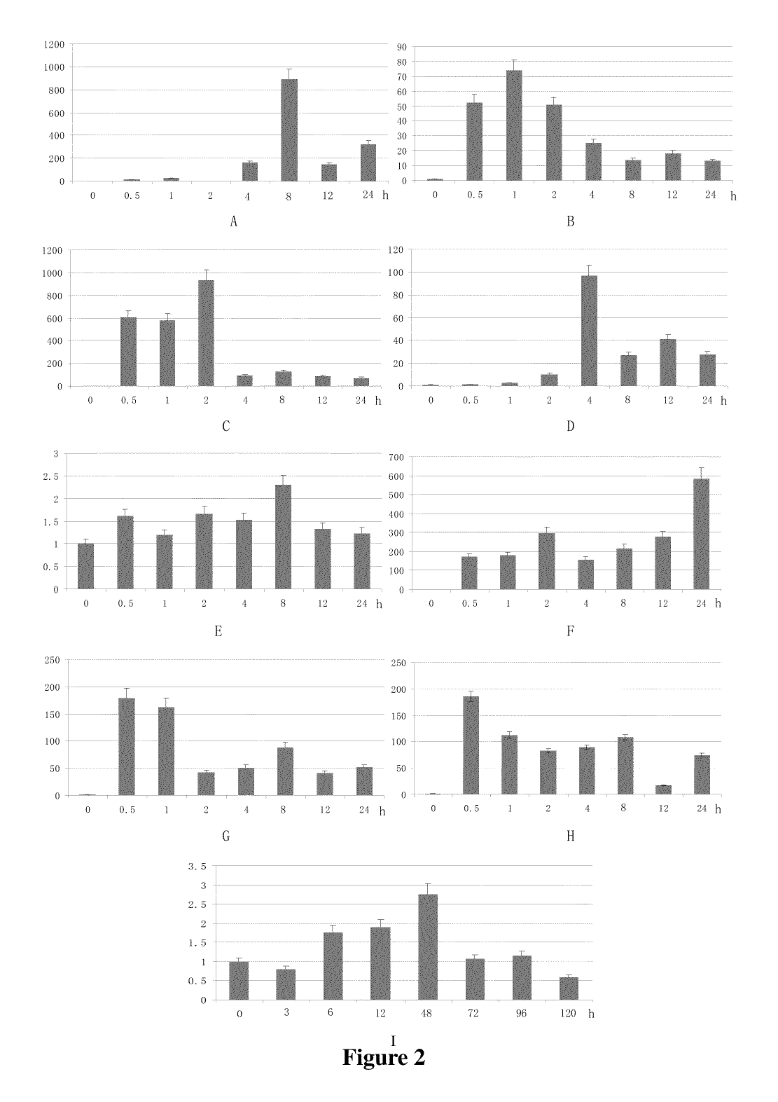

As shown in FIG. 2, TaDREB4B responded to various stresses and hormones.

### Example 3

### Activation Properties of TaDREB4B

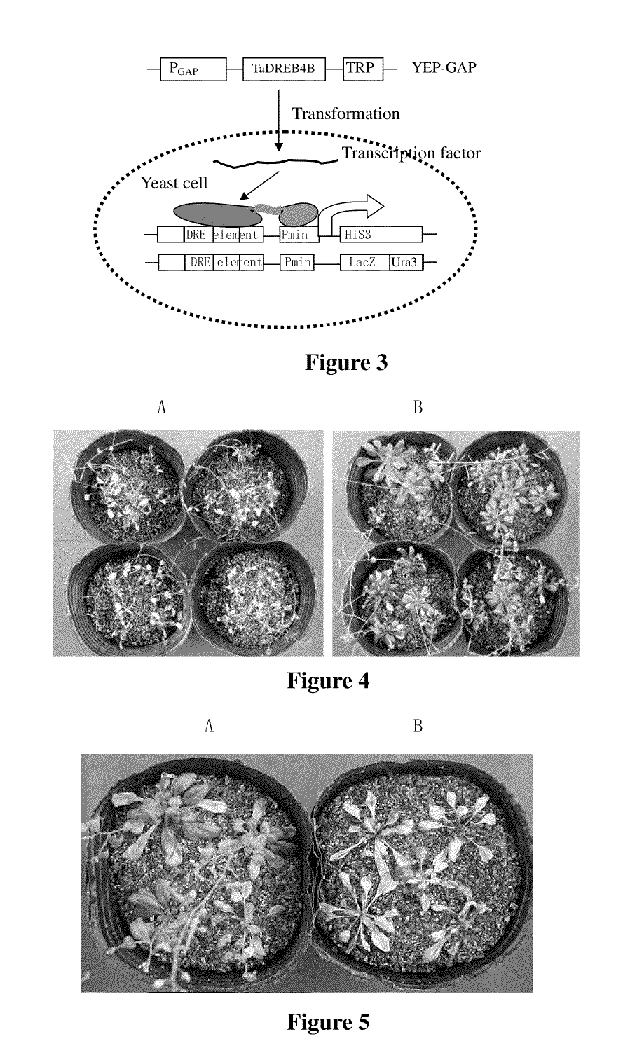

The yeast one-hybrid system was used to prove the main principle of the activation properties of a transcription factor, as shown in FIG. 3. DRE cis-acting element and mutant DRE cis-acting element were respectively constructed in pHISi-1 vector and pLacZi vector, upstream of the basal promoter Pmin (minimal promoter); downstream of the Pmin promoter was connected to reporter genes (His3, LacZ and URA3). Upon an expression vector YEP-GAP (having no activation function) connected with a target gene encoding a transcription factor is transformed to yeast cells connected with the DRE cis-acting element and mutant DRE cis-acting element, respectively, if the reporter gene in the yeast cell connected with the mutant DRE cis-acting element is unable to express while the reporter gene in the yeast cell connected with specific DRE cis-acting element is able to express, it indicated that the transcription factor is able to bind with DRE cis-acting element and has an activation function, which activates the Pmin promoter and promotes expression of the reporter gene; thereby proving the binding specificity in vivo and activation function of the target transcription factor.

YEP-GAP: the Institute of Crop Science, Chinese Academy of Agricultural Sciences promises to provide it to the public; Reference: Liu Q, Kasuga M, Sakuma Y, Abe H, Miura S, Yamaguchi-Shinozaki K, Shinozaki K. Two transcription factors, DREB1 and DREB2, with an EREBP/AP2 DNA binding domain separate two cellular signal transduction pathways in drought- and low-temperature-responsive gene expression, respectively, in Arabidopsis, Plant Cell 1998 August; 10(8):1391-1406.

YPD liquid medium: Bacto-Yeast Extract, 10 g/L, Bacto-Peptone, 20 g/L, adjusted to 5.8 in pH, sterilized at 121° C./15 min, and added with 40% Glucose after temperature dropping to 60° C., final concentration, 20 g/L.

SD/His⁻/Ura⁻/Trp⁻ selective medium: yeast nitrogen base free of amino acid, 6.7 g/L, auxotrophic mixture (drop-out media without His/Ura/Trp), 100 ml, Bacteriological agar, 20 g/L, adjusted to 5.8 in pH, sterilized at 121° C./15 min, and added with 40% Glucose after temperature dropping to 60° C., final concentration, 20 g/L.

Auxotrophic mixture: (10×): L-Isoleucine, 300 mg/L; L-Valine, 1500 mg/L; L-Adenine, 200 mg/L; L-Arginine, 200 mg/L; L-Histidine Hcl monohydrate, 200 mg/L; L-Leucine, 1000 mg/L; L-Lysine Hcl, 300 mg/L; L-Methionine, 200 mg/L; L-Phenylalanine, 500 mg/L; L-Threonine, 2000 mg/L; L-Tyrosine, 300 mg/L.

1×PEG/LiAc: 50% PEG3350, 8 ml; 10×TE buffer, 1 ml; 10×LiAc, 1 ml.

10×TE Buffer: 100 mM Tris-Hcl, 10 mM EDTA, pH=7.5, autoclave sterilized at 121° C., and stored at room temperature.

1×TE/LiAc: 10×TE buffer, 1 ml; 10×LiAc, 1 ml; ddH₂O, 8 ml.

Z Buffer: Na₂HPO₄.7H₂O, 16.1 g/L; NaH₂PO₄.H₂O, 5.5 g/L; KCl, 0.75 g/L; MgSO₄.7H₂O, 0.246 g/L; adjusted to pH 7.0, sterilized at 121° C./15 min, and stored at 4° C.

X-gal Stock Solution: X-gal was dissolved with N,N-dimethyl-formamide (DMF) to have a final concentration of 20 mg/ml, and stored at −20° C.

Z buffer with X-gal, 100 ml, formulated immediately before use: Z buffer, 98 ml, β-mercaptoethanol, 0.27 ml, X-gal stock solution, 1.67 ml.

10×LiAc: Clontech Company.

### I. Construction of Recombinant Expression Vector

1. Obtaining of TaDREB4B gene

Primers, TaDREB4B-EI and TaDREB4B-XI were designed based on the sequence of TaDREB4B gene; the terminals of the primers were introduced with digestion sites of EcoRI and XhoI, respectively; PCR amplification was conducted with the cDNA of Xiaobaimai as the template to give the TaDREB4B gene.

TaDREB4B-EI:
5′-GGGGAATTCATGACGGTAGATCGGAAGGAC-3′;
TaDREB4B-XI:
5′-GGGCTCGAGATGGTTTGGCCGCCGCAAAG-3′.

PCR amplification products were detected via a 1.2% agarose gel electrophoresis.

Agarose Gel DNA Purification Kit Ver.2.0 (TaKaRa Company, Code No.: DV807A) was used to recover and purify the PCR products of about 1.1 Kb.

### 2. Construction of Recombinant Expression Vector

\hat{1} PCR products recovered and purified in step 1 were digested with restriction endonucleases, EcoRI and XhoI, and the digestion products were recovered;

\hat{2} the expression vector YEP-GAP was digested with restriction endonucleases EcoRI and XhoI, and vector backbones were recovered;

\hat{3} the digestion products of step \hat{1} were ligated to the vector backbones of step \hat{2};

\hat{4} ligation products of step \hat{3} were electro-transformed into a JM109 strain (purchased from the Clontech Company), cultivated at 37° C. overnight, from which, positive clones were selected for sequencing; the sequencing result indicated that a recombinant plasmid YEP-GAP-TaDREB4B (a DNA fragment represented by nucleotides at positions 128-1193 starting from the 5′ end of SEQ ID NO. 2 in the Sequence Listing was inserted into YEP-GAP between the digestion sites of EcoRI and XhoI) was obtained.

### II. Verification of Binding Specificity In Vivo and Activation Properties of TaDREB4B

### 1. Construction of Yeast Reporter

### (1) Construction of Normal Yeast Dual Reporters

DNA fragment A (containing 4 DRE elements): 5′-GAATTC-DRE-DRE-DRE-DRE-GTCGAC-3′ (core sequence of DRE: TACCGACAT). The DNA fragment A has a nucleotide sequence as shown in SEQ ID NO. 3 of the Sequence Listing.

DNA fragment A was constructed into pHis-1 vector (MATCHMAKER One-Hybrid System, Clontech Company), upstream of the Pmin_{His3} promoter, giving a recombinant vector pHis-1-DRE, which was cut with endonucleases XhoI and NcoI into linear shapes.

DNA fragment A was constructed into pLacZi vector (MATCHMAKER One-Hybrid System, Clontech Company), upstream of the P_{CYCI} promoter, giving a recombinant vector pLacZi-DRE, which was cut with endonucleases XhoI and NcoI respectively into linear shapes.

Firstly, the linear pHis-1-DRE vector was transformed into a yeast cell (YM4271 plant line, MATCHMAKER One-Hybrid System, Clontech Company) to give a yeast transformant that is able to normally grow on a SD/His⁻ medium. Next, transformation of linear pLacZi-DRE-containing vector was continued, with this yeast transformant as the host cell. Therefore, normal yeast dual reporters containing pHis-1-DRE and pLacZi-DRE were selected and obtained on a SD/His⁻/Ura⁻ medium lack of both histidine and uracil.

### (2) Construction of Mutant Yeast Dual Reporters

DNA fragment B (containing 4 MDRE elements): 5′-GAATTC-MDRE-MDRE-MDRE-MDRE-GTCGAC-3′ (MDRE: core sequences, CCGAC, of the four MDRE elements were mutated into TTTTT). DNA fragment B has a nucleotide sequence as shown in SEQ ID NO. 4 of the Sequence Listing.

Using the same methods as described in step (1), mutant yeast dual reporters were obtained with DNA fragment A replaced by DNA fragment B.

2. Transformation of Yeast with PEG/LiAc Method and Analysis of the Results

(1) Yeast strains (YM4271 plant line) were inoculated into a 1 ml YPD liquid medium, strongly oscillated for 2 min, and the resultant suspension, after clumps dispersed, was transferred into a erlenmeyer flask containing 50 ml YPD liquid medium, and shaken at 30° C./250 rpm overnight; OD600 was determined to be 1.7-1.8 (about 4×10/mL by counting);

(2) 30 ml of the overnight culture of step (1) was ligated into 300 ml fresh YPD medium, cultivated at 30° C./250 rpm for about 3 h (until OD600=0.5±0.1), and centrifuged at 1000 g at room temperature for 5 min; the supernatant was discarded to collect strains, which were suspended with ½ volume of 1×TE, and centrifuged at 1000 g for 5 min;

(3) the supernatant was sucked out and discarded, while the remains were suspended with 1.5 ml of freshly formulated 1×TE/LiAc solution, oscillated and mixed for use;

(4) 0.1 ml yeast competent cells was taken and used to transform, into which the following solutions were added in the order of: 0.1 μg YEP-GAP-TaDREB4B, 0.1 mg ssDNA (salmon sperm DNA, Sigma), 0.6 ml PEG/LiAc; the mixture was subjected to a high speed oscillation for 1 min, cultivated while shaken at 30° C./200 rpm for 30 min;

(5) 70 ul DMSO (sigma #D8779) was added into the mixture, which was gently placed upside down and mixed, heat shocked at 42° C. for 30 min while gently oscillated, ice bathed for 2 min and centrifuged at 1000 g at room temperature for 5 min;

(6) the supernatant was sucked out and discarded, and the remaining cells were suspended by addition of 0.5 ml 1×TE buffer;

(7) the suspension was taken with a inoculating loop by dipping, and cultivated respectively on SD/His⁻/Ura⁻/Trp⁻ selective mediums containing 0, 15 mmol/L 3-AT by drawing lines thereon.

(8) one half of the plate was used to cultivate normal yeast dual reporters, the other half was used to cultivate mutant yeast dual reporters so as to make comparative analysis.

(9) the plate was placed upside down in an incubator, and cultivated at 30° C. for 3-4-d.

(10) it was found that both normal yeast reporters and mutated yeast reporters grown on the SD/His⁻/Ura⁻/Trp⁻ medium plate containing 0 mmol/L 3-AT, whereas, the mutated yeast reporter has a significantly smaller diameter; while on the SD/His⁻/Ura⁻/Trp⁻ medium plate containing 15 mmol/L 3-AT, the normal yeast reporters were able to normally grow but the mutated yeast reporters were inhibited to grow.

### 3. Detection of Galactosidase Activity

(1) Colonies of Both Normal Yeast Reporters and Mutated Yeast Reporters were picked up from the SD/His⁻/Ura⁻/Trp⁻ medium plate containing 0 mmol/L 3-AT, transferred into a YPD liquid medium, cultivated at 30° C. while oscillated; after growing to the late phase of logarithmic growth, 1.5 ml bacteria solution was taken for a centrifugation at 3000 rpm for 30 s;

(2) the supernatant was discarded, and the liquids were removed from the tube; the centrifuge tube was quickly frozen in liquid nitrogen for 10 min, taken out to make it thaw naturally, into which 50 ul Z/X-gal solution was added, and incubated at 30° C.; as a result, the normal yeast reporters turned blue within 6-8h while the mutated yeast reporters kept white within 12 h, suggesting that the transcription factor TaDREB4B is able to bind with DRE cis-acting element and has an activation function, which activates the Pmin promoter and promotes expression of the reporter gene; thereby proving the binding specificity in vivo and activation function of TaDREB4B.

### Example 4

### TaDREB4B Improved the Drought, Salinity and High Temperature Tolerances of Arabidopsis

### I. Construction of Recombinant Expression Vector

### 1. Cloning of the TaDREB4B Gene

Primer pair (TaDREB4B-121F and TaDREB4B-121R) was designed based on the sequence of TaDREB4B gene; the terminals of the primers were introduced with digestion recognition sites of BamHI and XhoI, respectively; PCR was conducted with the cDNA of Xiaobaimai as the template to amplify TaDREB4B.

TaDREB4B-121F:
5′-GGGGGATCCATGACGGTAGATCGGAAGGAC-3′;
TaDREB4B-121R:
5′-GGGCTCGAGATGGTTTGGCCGCCGCAAAG-3′.

PCR amplification products were detected via a 1.2% agarose gel electrophoresis. Agarose Gel DNA Purification Kit Ver.2.0 (TaKaRa Company, Code No.: DV807A) was used to recover and purify the bands of about 1.1 Kb.

### 2. Construction of Recombinant Expression Vector

\hat{1} PCR products recovered and purified in step 1 were digested with restriction endonucleases, BamHI and XhoI, and the digestion products were recovered;

\hat{2} pBI121 (purchased from Clontech Company) was digested with restriction endonuclease BamHI and XhoI, and vector backbones were recovered;

\hat{3} the digestion products of step \hat{1} were ligated to the vector backbones of step \hat{2};

\hat{4} the ligation products of step \hat{3} were electro-transformed into a TOP10 strain (purchased from TIANGEN BIOTECH (BEIJING) CO. LTD.), cultivated at 37° C. overnight, from which, positive clones were picked up for sequencing; the sequencing result indicated that a recombinant plasmid pBI121-TaDREB4B (a DNA fragment represented by nucleotides at positions 128-1193 starting from the 5′ end of SEQ ID NO. 2 in the Sequence Listing was inserted into pBI121 between the digestion sites of BamHI and XhoI) was produced.

### II. Obtaining of Transgenic Plants

1. Recombinant plasmid pBI121-TaDREB4B was used to transform Agrobacterium C58 (purchased from Clontech Company) to give a recombinant Agrobacterium;

2. the recombinant Agrobacterium was inoculated in a YEP liquid medium, cultivated at 28, 3000 rpm, for about 30 h;

3. the bacteria solution of step 2 was transferred into a YEP liquid medium (containing 50 μg/L kanamycin and 50 μg/L Rifampicin), and cultivated at 28° C., 300 rpm, for about 14 h (OD600 of the bacteria solution reached 1.5-3.0);

4. thalluses were collected, centrifuged at 4, 4000 g, for 10 min, and diluted with 10% sucrose (containing 0.02% silwet) until the OD600 reached about 0.8-1.0;

5. the whole plant of Arabidopsis (Columbia ecotype Col-0, purchased from SALK Company) together with the flowerpot were reversely put into a container filled with the bacteria solution of step 4, dipped for about 50 s, after that, the flowerpot was taken out, laid on its side on a tray, and covered with a black plastic cloth, which, after 24 h, was lifted; and then, the flowerpot was placed upright, and the plants were subjected to normal cultivation under light, harvested for seeds of T₁ generation, and screened for positive plants with kanamycin (having a concentration of 50 μg/L kanamycin). The positive plants were detected via PCR, and the result showed that transgenic plants (transgenic plants of TaDREB4B gene) were obtained.

T₂ generation denotes seeds produced by T₁ generation via selfing and plants grown from these seeds; and T₃ generation denotes seeds produced by T₂ generation via selfing and plants grown from these seeds.

III. Obtaining of Control Plant Transformed with Empty Vector

By employing the same methods as described in step II, plasmid pBI121 was used to transform Agrobacterium to give a recombinant Agrobacterium; and recombinant Agrobacterium was used to transform Arabidopsis Col-0 to give a control plant transformed with empty vector.

### IV. Stress Tolerance Identification of Transgenic Plants

Transgenic plants of T₃ generation, control plants transformed with empty vector of T₃ generation and plants of Arabidopsis Col-0 (60 plants each) were subjected to identification for drought tolerance, salinity tolerance and high temperature tolerance, respectively. Experiments were conducted in triplicate with an average value as the final result.

### 1. Drought Tolerance

Normally grown seedlings of 3 weeks were not watered for 27 consecutive days; and on day 28, survival rate was counted. All plants of Arabidopsis Col-0 died, but 90% of the transgenic plants survived and were able to grow normally (see FIG. 4, A: Arabidopsis Col-0; B: transgenic plant). Control plants transformed with empty vectors have a phenotype identical with that of Arabidopsis Col-0, and a survival rate that is not significantly different from that of Arabidopsis Col-0.

### 2. Salinity Tolerance

Normally grown seedlings of 3 weeks were irrigated with 400 mM NaCl, then, transferred to a normal flowerpot and normally administrated. After two weeks, survival rate was counted. Nearly all plants of Arabidopsis Col-0 died, but 55% of the transgenic plants still survived and were able to grow normally (see FIG. 5, A: transgenic plant; B: Arabidopsis Col-0). Control plants transformed with empty vector have a phenotype identical with that of Arabidopsis Col-0, and a survival rate that is not significantly different from that of Arabidopsis Col-0.

### 3. High Temperature Tolerance

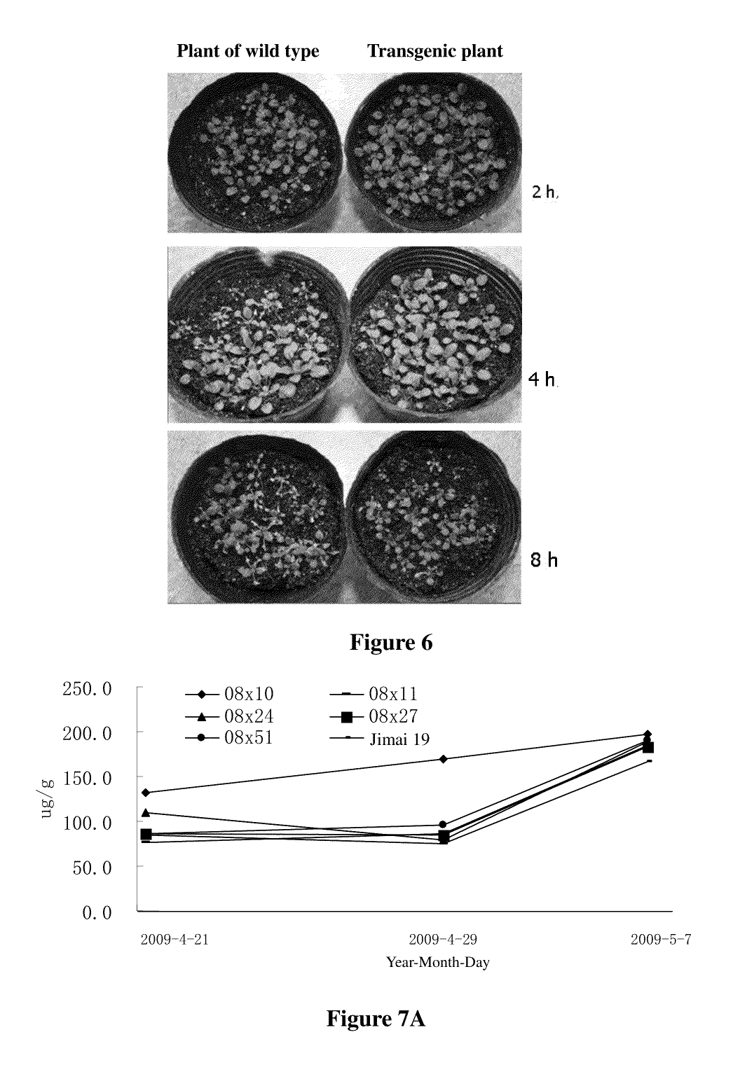

Normally grown seedlings of 2 weeks were treated with high temperature (43° C.) for 2 h, 4 h, 8 h, respectively, and then, resumed to grow at normal temperature for one week before counting survival rate. After high temperature treatment for 2 h, both Arabidopsis Col-0 and transgenic plants have a survival rate of 100%; after high temperature treatment for 4 h, plants of Arabidopsis Col-0 have a survival rate of 50%, and 100% of transgenic plants survived and were able to grow normally; after high temperature treatment for 8 h, plants of Arabidopsis Col-0 have a survival rate of 30%, and 55% of transgenic plants survived and were able to grow normally (see FIG. 6). Control plants transformed with empty vector have a phenotype identical with that of Arabidopsis Col-0, and a survival rate that is not significantly different from that of Arabidopsis Col-0.

### Example 5

### TaDREB4B Improved Drought Resistance and Stress Tolerance to Pathogenic Bacteria of Wheat

### I. Construction of Recombinant Expression Vector

### 1. Obtaining of TaDREB4B Gene

Primer pair (TaDREB4B-121F and TaDREB4B-121R) was designed based on the sequence of TaDREB4B gene; the terminals of the primers were introduced with digestion sites of SmaI and SpeI, respectively; PCR was conducted with the cDNA of Xiaobaimai as the template to amplify TaDREB4B gene.

TaDREB4B-121F:
5′-TTTCCCGGGATGACGGTAGATCGGAAGGAC-3′;
TaDREB4B-121R:
5′-GGGACTAGTATGGTTTGGCCGCCGCAAAG-3′.

PCR amplification products were detected via a 1.2% agarose gel electrophoresis.

Agarose Gel DNA Purification Kit Ver.2.0 (TaKaRa Company, Code No.: DV807A) was used to recover and purify the PCR products of about 1.1 Kb.

### 2. Construction of Recombinant Expression Vector

\hat{1} PCR products recovered and purified in step 1 were digested with restriction endonucleases, SmaI and SpeI, and the digestion products were recovered;

\hat{2} pAHC25 (purchased from Beijing BioDee BioTech Corporation Ltd.) was digested with restriction endonuclease SmaI and SpeI, and vector backbones were recovered;

\hat{3} the digestion products of step \hat{1} were ligated to the vector backbones of step \hat{2};

\hat{4} the ligation products of step \hat{3} were electro-transformed into a TOP10 strain (purchased from TIANGEN BIOTECH (BEIJING) CO. LTD.), cultivated at 37° C. overnight, from which, positive clones were picked up for sequencing; the sequencing result indicated that a recombinant plasmid pAHC25-TaDREB4B (a DNA fragment represented by nucleotides at positions 128-1193 starting from the 5′ end of SEQ ID NO. 2 in the Sequence Listing was inserted into pAHC25 between the digestion sites of SmaI and SpeI) was produced.

### II. Obtaining of Transgenic Plant

1. Transformation of Wheat Callus with Gene Gun

Field grown wheat (Jimai 19; purchased from Shandong Academy of Agricultural Science) was taken off its immature embryo 14 days after pollination, which was inoculated on a SD2 medium, callus was induced at 26° C. under dark condition, and prepared, after 7-10 days, for gene gun bombardment.

Appropriate amount of golden powder (1.0 μm) suspension (60 μg/gun) was mixed with pAHC25-TaDREB4B, oscillated at 4° C. for 10 min, centrifuged at 14000 rpm for 5 min to remove supernatant, anhydrous ethanol was added (added in 10 μl/gun), and prepared for gene gun bombardment.

PDS-1000/He gene gun (produced by Bia-Rod Company) was used to bomb the callus induced by wheat immature embryos. A splittable film of 1100 Psi was selected to conduct a bombardment on materials. The bombed callus continued to be cultivated on the original osmotic pressure medium for 16-18h, and then, transferred into a SD2 medium (MM medium can also work) free of selective agent, and resumed to be cultivated under dark condition (26° C.) for 2 weeks. After two weeks, callus was transferred on the first screening medium (½MS+zeatin, 0.5 mg/L, +2% sucrose+bilanafos sodium, 3 mg/L; or ½ MS+α-naphthyl acetic acid, 1 mg/L, +6-furfuryl-aminopurine, 0.5 mg/L, +2% sucrose+bilanafos sodium, 3 mg/L also works), and cultivated at 24° C. (10h of light per day) for four weeks so as to screen differentiation. When green shoots were differentiated from the callus, they were transferred onto a hormone-free medium (½MS+bilanafos sodium, 4 mg/L) until seedlings elongated (with the same light and temperature as described above) to 1-2 cm (required about 4 weeks). The anti-bilanafos sodium, regenerated plants were shifted into a plantlet strengthening medium (½MS+auxin, 0.5 mg/L, +paclobutrazol, 0.5 mg/L) to strengthen the seedlings, then shifted into a nutrition bowl when they growing to appropriate sizes (seedling height, 6-8 cm, good roots), cultivated at about 15° C. under light, and placed into a greenhouse after the seedlings were strengthened.

The resultant positive seedlings were subjected to molecular identification, the result showed that transgenic plants (T₀ generation) were obtained. T₁ generation denotes seeds produced by T₀ generation via selfing and plants grown from these seeds; T₂ generation denotes seeds produced by T₁ generation via selfing and plants grown from these seeds; T₃ generation denotes seeds produced by T₂ generation via selfing and plants grown from these seeds; T₄ generation denotes seeds produced by T₃ generation via selfing and plants grown from these seeds; T₅ generation denotes seeds produced by T₄ generation via selfing and plants grown from these seeds; T₆ generation denotes seeds produced by T₅ generation via selfing and plants grown from these seeds.

III. Obtaining of Control Plants Transformed with Empty Vector

Using the same methods as described in step II, control plants transformed with empty vector were prepared with pAHC25-TaDREB4B replaced by pAHC25.

### IV. Drought Resistance of Transgenic Plants

T₆ generation plants of five plant lines (08×10, 08×11, 08×24, 08×27, 08×51) of the transgenic plants, T₆ generation plants of control plants transformed with empty vector and Jimai 19 (30 plants each) were planted in field at October, 2008. Drought resistance indexes were determined in 2009. Determination method for proline content can be found in the Reference: Zhang Dian-zhong, Wang Pei-hong, Zhao Hui-xian, Determination of the Content of Free Proline in Wheat Leaves, 1990(4): 62˜65. Determination method for the content of total soluble sugar can be found in the Reference: Zou Qi, Guidance of Plant Physiological and Biochemical Experiment, Beijing: China Agriculture Press, 1995. Determination method for peroxidase activity (POD enzyme activity) can be found in the Reference: Xu Lang-lai, Ye Mao-bing, Determination Method for Peroxidase Activities through Continuous Recoding, Journal of Nanjing Agricultural University, 1989, 12(3): 82-83; Chedf M, Aseelin A, Belenger R R. Defense Response Induced by Soluble Silicon in Cucumber Roots Infected by Pyshium spp. phytopathology, 1994, 84: 236-275. Photosynthetic rate (SPAD value) was determined by LI-6400 portable photosynthesis analyzer. Experiments were conducted in triplicate with an average value as the final result.

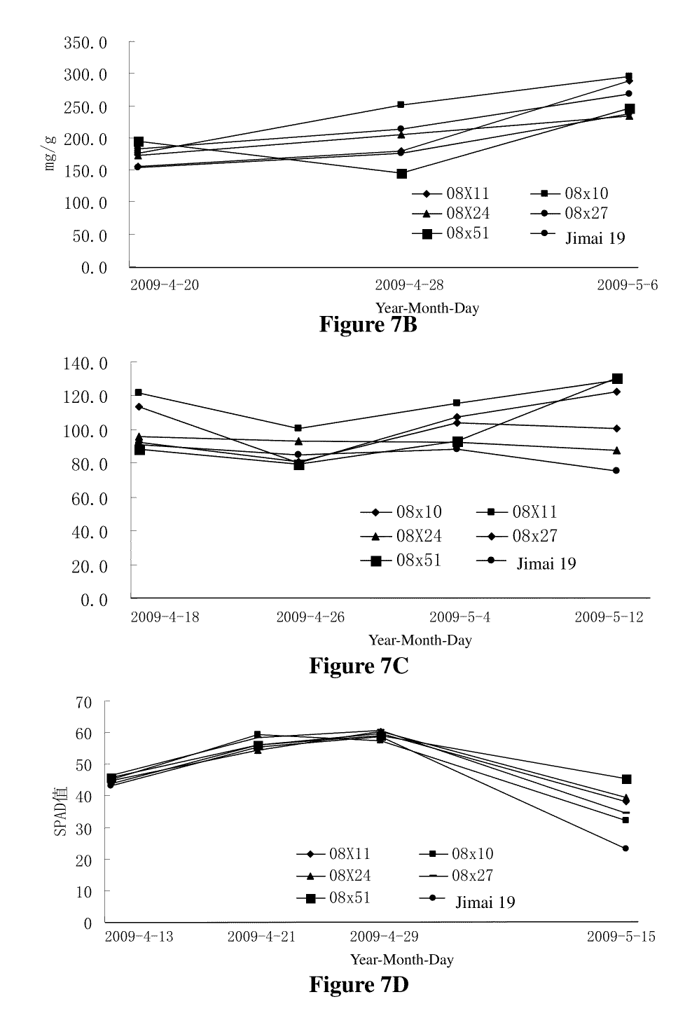

As shown in FIG. 7, the results indicated that: transgenic plant has a proline content (FIG. 7A), a content of total soluble sugar (FIG. 7B), a peroxidase activity (FIG. 7C), and a photosynthetic rate (FIG. 7D) significantly higher than that of Jimai 19, respectively. Control plant transformed with empty vector has a proline conten, a content of total soluble sugar, a peroxidase activity and a photosynthetic rate that are not significantly different from that of Jimai 19, respectively.

T₆ generation plants of eight plant lines (08×6, 08×10, 08×11, 08×18, 08×24, 08×27, 08×28, 08×36) of transgenic plants, T₆ generation plants of control plants transformed with empty vector and Jimai 19 (30 plants each) were planted in dryland at October, 2008. Indexes such as plant height, number of ears, number of grains per plant, grain weight per plant, 1000-grains weight and the like were determined in 2009. Experiments were conducted in triplicate with an average value as the final result. As shown in Table 2, the results indicated that: transgenic plant is significantly improved in grain weight per plant and 1000-grains weight as compared with Jimai 19; control plant transformed with empty vector is not significantly different from Jimai 19 in various indexes.

TABLE 2
main characteristic values of transgenic (DREB4 gene) wheat lines

| | | | number of | grain | |
|---|---|---|---|---|---|
| | plant height | number of | grains per | weight per | 1000-grains |
| plant lines | (cm) | ears | plant | plant (g) | weight (g) |
| 08X6 | 66.2 | 5.67 | 221.5 | 11.74 | 53.3 |
| 08X10 | 65.1 | 5.06 | 171.8 | 8.39 | 48.9 |
| 08X11 | 63.3 | 5.57 | 193.1 | 9.46 | 48.8 |
| 08X18 | 66.4 | 6.58 | 238.6 | 12.55 | 52.1 |
| 08X24 | 61.4 | 5.00 | 155.9 | 7.45 | 47.8 |
| 08X27 | 62.9 | 5.53 | 164.9 | 7.86 | 47.8 |
| 08X28 | 67.0 | 6.31 | 188.9 | 9.91 | 52.3 |
| 08X36 | 65.1 | 8.30 | 279.5 | 14.61 | 52.2 |
| DREB4B, | 64.7 | 6.00 | 201.78 | 10.03 | 50.4 |
| mean value | | | | | |
| Jimai 19 | 63.7 | 6.30 | 212.7 | 8.78 | 41.2 |

### V. Disease Resistance Cases of Transgenic Wheat

T₆ generation plants of transgenic plant line 08×18, T₆ generation plants of control plants transformed with empty vector and Jimai 19 (30 plants each) were planted in the field at October, 2008. The plants were transplanted into a greenhouse at February, 2009, and inoculated with pathogenic bacteria of powdery mildew E09 (microspecies 15, prevalent in Beijing region, with a toxicity profile: Vir1, 3a, 3b, 3c, 3e, 5, 6, 7, 8, 17, 19; purchased from Institute of Plant Protection, Chinese Academy of Agricultural Science) in late March. Two weeks later, plants were observed and photographed, and identified for disease resistance at the same time; the identification method can be found in the Reference: Xie Hao, Chen Xiao, Sheng Bao-qin, Xin Zhi-yong, Kong Fan Jing, Lin Zhi-shan, Ma You-zhi, Identification of Resistance to Powdery Mildew and Genetic Analysis of a New Wheat Line YW243, ACTA AGRONOMICA SINICA, 2001 27(6), 715-721.

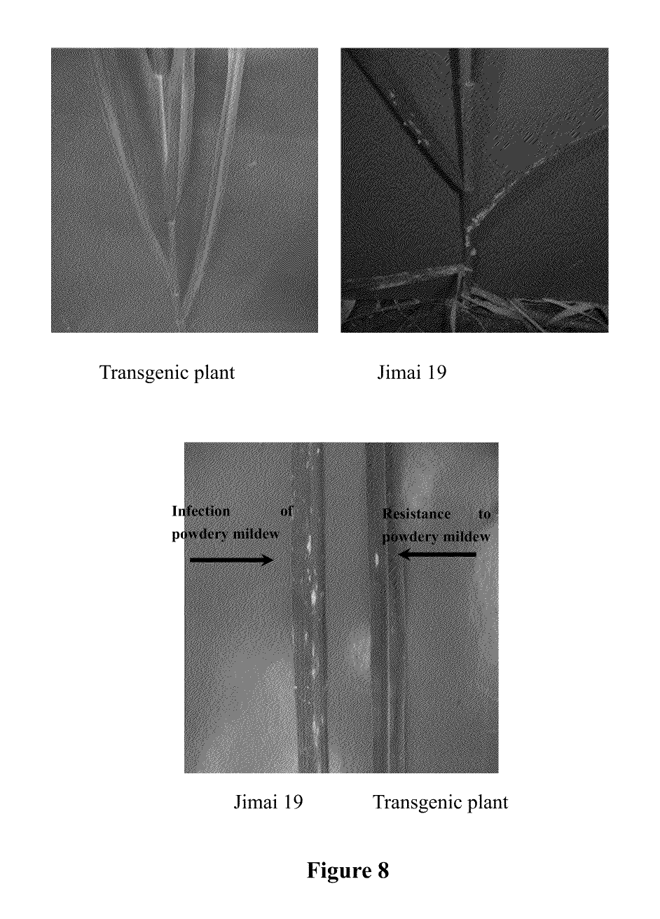

As shown in FIG. 8, the identification results of disease resistance indicate that number of disease lesions per unit area in the leaves of transgenic plant is significantly smaller than that of Jimai 19 and control plant transformed with empty vector, that is, transgenic plant has an improved resistance to the pathogenic bacteria of powdery mildew; control plant transformed with empty vector has a phenotype identical with that of the wild type, and a resistance to the pathogenic bacteria of powdery mildew that is not significantly different.

### INDUSTRIAL APPLICATION

The TaDREB4B of the present invention is expressed under the induction of drought, high salinity, high temperature, low temperature, pathogenic bacteria, ABA, ethylene, JA, and SA, and can specially regulate the transcriptional expression of a gene containing DRE/CRT cis element (core sequence: CCGAC), thereby enhancing the drought resistance, salinity tolerance, high temperature tolerance and resistance to pathogenic bacteria of powdery mildew of plant. The TaDREB4B of the present invention provides a basis for artificial control on expressions of stress resistance and stress tolerance related genes, which will play an important role in cultivating a plant improved in stress resistance and stress tolerance.

1-10. (canceled)

11. An isolated nucleic acid that encodes a protein with at least 90% amino acid sequence identity to SEQ ID NO: 1.

12. The isolated nucleic acid of claim 11, comprising a nucleotide sequence selected from the group consisting of:
(a) SEQ ID NO: 2;
(b) nucleotides 128-1168 of SEQ ID NO: 2;
(c) nucleotides 128-1193 of SEQ ID NO: 2; and
(e) a nucleotide sequence fully complementary to one or more of (a)-(c); and
(f) a nucleotide sequence which hybridizes under high stringency conditions with the nucleotide sequence of any one of (a)-(c).

13. An expression vector comprising the isolated nucleic acid sequence of claim 11.

14. The expression vector of claim 13, comprising a nucleotide sequence selected from the group consisting of:
(a) SEQ ID NO: 2;
(b) nucleotides 128-1168 of SEQ ID NO: 2; and
(c) nucleotides 128-1193 of SEQ ID NO: 2.

15. The expression vector of claim 13, selected from the group consisting of: YEP-GAP-TaDREB4B, pBI121-TaDREB4B and pAHC25-TaDREB4B.

16. An expression cassette comprising the nucleic acid sequence of claim 11.

17. A plant cell comprising the nucleic acid of claim 11.

18. The plant cell of claim 17, comprising a nucleotide sequence selected from the group consisting of:
(a) SEQ ID NO: 2;
(b) nucleotides 128-1168 of SEQ ID NO: 2; and
(c) nucleotides 128-1193 of SEQ ID NO: 2.

19. A plant comprising the nucleic acid of claim 11.

20. The plant of claim 19, comprising a nucleotide sequence selected from the group consisting of:
(a) SEQ ID NO: 2;
(b) nucleotides 128-1168 of SEQ ID NO: 2; and
(c) nucleotides 128-1193 of SEQ ID NO: 2.

21. The plant of claim 20, wherein the plant exhibits enhanced stress tolerance when compared to a control plant.

22. The plant of claim 21, wherein the stress tolerance includes one or more selected tolerances from the group consisting of: abiotic stress tolerance and disease resistance.

23. The plant of claim 21, wherein the abiotic stress tolerance is one or more tolerances selected from the group consisting of: drought tolerance, salinity tolerance and tolerance to high temperature, and wherein the disease resistance is resistance to powdery mildew.

24. The plant of claim 19, wherein the plant is a monocotyledonous plant.

25. The plant of claim 19, wherein the plant is a dicotyledonous plant.

26. The plant of claim 25, wherein the plant is a member of the genus Arabidopsis or wheat.

27. An isolated bacterial strain comprising the nucleic acid of claim 11.

28. The isolated bacterial strain of claim 27, wherein the bacterial strain is Agrobacterium tumerfaciens.

29. An isolated protein comprising an amino acid sequence with at least 90% amino acid sequence identity to SEQ ID NO: 2.

30. The isolated protein of claim 29, comprising SEQ ID NO: 2.

31. The isolated protein of claim 29, consisting of SEQ ID NO: 2.

32. A method for generating a plant with higher stress tolerance than a control plant, the method comprising:
introducing into one or more cells of the plant an isolated nucleic acid that encodes a protein with at least 90% amino acid sequence identity to SEQ ID NO: 1.


SEQUENCE LISTING (21 sequences)

SEQ ID NO: 1 - protein, 346 aa, Triticum aestivum
MTVDRKDAEAAAAAATPFEIPALQPGRKKRPRRSRDGPNSVSETIRRWKEVNQQLEHDPQGAKRARKPPAKGSKKGCMQGKGGPENTQCGFRGVRQRTWGKWVAEIREPNRVSRLWLGTFPTAEDAARAYDEAARAMYGALARTNFPAHPAQAPAVALPAAIEGVVRGASASCESTTTSANHSDVASNLPRQAQALEIYSQPDVLESTESVVLESVEHYSHKDTVPDAGSSIARSTSEEDVFEPLEPISSLPDGEADGFDIEELLRLMEADPIEVEPVTGGSWNCGTNTGVEMGLQEPLYLDGLDQGMLEGMLQADYPYPMWISEDRAMRNPAFHDAEMSEFFEGL

SEQ ID NO: 2 - DNA, 1494 bp, Triticum aestivum
gccgacttttctttctctcctcctccacgcctctccccaactctctatccaagtccacgcggcgaagaaaccaggcgacaagattgcgaacgctagatatctggacccgatccggatcgggccggccatgacggtagatcggaaggacgccgaggcggcggcggcggcggcgacgcccttcgagatcccggcgctccagcctggaaggaaaaagcgacctcggagatcacgtgatgggcctaattcagtctctgaaacgatcaggcgatggaaagaagtgaaccaacaactggagcatgatccacagggtgcaaagagggcgaggaagccacctgcaaagggttcaaagaagggctgtatgcaggggaaaggaggacctgagaatacacaatgtggattccgtggtgtaaggcaacgtacttgggggaagtgggttgctgaaattcgggagccaaatcgggtgagcaggctctggttgggaacgttccccactgctgaggatgctgcccgtgcttatgacgaggcagcaagagcaatgtatggcgccctggctcgtaccaacttccctgcgcatcctgcacaagctcctgctgtggctctaccagcggcaattgaaggtgttgtacgtggtgcttcagcatcatgcgagtctactacaacatcagccaaccactcagatgttgcttctaacttgccgcgacaagctcaagctcttgagatttactcccagccagatgtgcttgagtccaccgaatcagttgtgctggagtctgtcgagcattacagccataaagacactgttcctgatgctggctcaagcattgcaaggagcacatccgaagaggatgtgttcgagccattggagcctatttccagtttgccggatggggaagcagacggttttgatatagaagaattactgagattgatggaagccgacccaattgaagttgagccggtcactgggggctcctggaattgtggaaccaacactggcgtggagatgggcctgcaggaacctctctacctggatggcttggaccaaggcatgctggagggcatgctgcaagctgattatccttacccaatgtggatatcagaggatcgggccatgcgcaaccctgccttccatgatgctgagatgagcgagttcttcgaagggttgtgatccccctttgcggcggccaaaccatgtctatggtgtttggtcggcttgcccttcggtgtccgctgctgtgctccaatgaagatcaaatggtggaccagaagattggattcctctgcagaactaataagctcctagctagttttttgtgcttcgtttgtagttctgttaggcatgggaactcttctctgtttcgatgtttcttgtgataagaaaccttgattgtgcatcacgatctttggaaggtgggaaaagaaaatgtgaaaatgcatttccgtggcaaaaaaaaaaaaaaaaaaaaaaaaaaaaaaaaaaaaaaaaaaaaaaaa

SEQ ID NO: 3 - DNA, 48 bp, Artificial Sequence
  Description of Artificial Sequence Synthetic oligonucleotide
gaattctaccgacattaccgacattaccgacattaccgacatgtcgac

SEQ ID NO: 4 - DNA, 48 bp, Artificial Sequence
  Description of Artificial Sequence Synthetic oligonucleotide
gaattctatttttattatttttattatttttattatttttatgtcgac

SEQ ID NO: 5 - protein, 5 aa, Artificial Sequence
  Description of Artificial Sequence Synthetic peptide
RRRRR

SEQ ID NO: 6 - protein, 6 aa, Artificial Sequence
  Description of Artificial Sequence Synthetic 6xHis tag
HHHHHH

SEQ ID NO: 7 - protein, 8 aa, Artificial Sequence
  Description of Artificial Sequence Synthetic peptide
DYKDDDDK

SEQ ID NO: 8 - protein, 8 aa, Artificial Sequence
  Description of Artificial Sequence Synthetic peptide
WSHPQFEK

SEQ ID NO: 9 - protein, 10 aa, Artificial Sequence
  Description of Artificial Sequence Synthetic peptide
EQKLISEEDL

SEQ ID NO: 10 - DNA, 21 bp, Artificial Sequence
  Description of Artificial Sequence Synthetic primer
accgcggtgtgaggcagagga

SEQ ID NO: 11 - DNA, 24 bp, Artificial Sequence
  Description of Artificial Sequence Synthetic primer
tgagaagttgacacgtgctttggc

SEQ ID NO: 12 - DNA, 21 bp, Artificial Sequence
  Description of Artificial Sequence Synthetic primer
gatgtgttcgagccattggag

SEQ ID NO: 13 - DNA, 21 bp, Artificial Sequence
  Description of Artificial Sequence Synthetic primer
tggtccaagccatccaggtag

SEQ ID NO: 14 - DNA, 18 bp, Artificial Sequence
  Description of Artificial Sequence Synthetic primer
ctccctcacaacaaccgc

SEQ ID NO: 15 - DNA, 22 bp, Artificial Sequence
  Description of Artificial Sequence Synthetic primer
taccaggaacttccataccaac

SEQ ID NO: 16 - DNA, 30 bp, Artificial Sequence
  Description of Artificial Sequence Synthetic primer
ggggaattcatgacggtagatcggaaggac

SEQ ID NO: 17 - DNA, 29 bp, Artificial Sequence
  Description of Artificial Sequence Synthetic primer
gggctcgagatggtttggccgccgcaaag

SEQ ID NO: 18 - DNA, 30 bp, Artificial Sequence
  Description of Artificial Sequence Synthetic primer
gggggatccatgacggtagatcggaaggac

SEQ ID NO: 19 - DNA, 30 bp, Artificial Sequence
  Description of Artificial Sequence Synthetic primer
tttcccgggatgacggtagatcggaaggac

SEQ ID NO: 20 - DNA, 29 bp, Artificial Sequence
  Description of Artificial Sequence Synthetic primer
gggactagtatggtttggccgccgcaaag

SEQ ID NO: 21 - protein, 278 aa, Triticum aestivum
METGGSKREGDCPGQERKKKVRRRSTGPDSVAETIKKWKEENQKLQQENGSRKAPAKGSKKGCMAGKGGPENSNCAYRGVRQRTWGKWVAEIREPNRGNRLWLGSFPTAVEAARAYDDAARAMYGAKARVNFSEQSPDANSGCTLAPPLPMSNGATAASHPSDGKDESESPPSLISNAPTAALHRSDAKDESESAGTVARKVKKEVSNDLRSTHEEHKTLEVSQPKGKALHKAANVSYDYFNVEEVLDMIIVELSADVKMEAHEEYQDGDDGFSLFSY